(19) United States
(12) Patent Application Publication
(10) Pub. No.: US 2022/0304963 A1
(43) Pub. Date: Sep. 29, 2022

(54) COMPOSITION HAVING AN OPTIMIZED FATTY ACID EXCIPIENT PROFILE

(71) Applicant: Basil Shaaban, Berut (LB)

(72) Inventor: Basil Shaaban, Berut (LB)

(21) Appl. No.: 17/545,967

(22) Filed: Dec. 8, 2021

Related U.S. Application Data
(63) Continuation-in-part of application No. 16/048,023, filed on Jul. 27, 2018, now abandoned.

(51) Int. Cl. A61K 31/194; A23L 29/00

(52) U.S. Cl. CPC A61K 31/194; A23L 29/04

(57) ABSTRACT

A composition of matter for enhancing delivery of bioactive compounds, particularly cannabinoids into the human and animal organisms. The invention enhanced oral, transmucosal and transdermal delivery of a bioactive compound including cannabinoids through the use of an excipient having a desired ratio of fatty acids, and the inclusion of terpenes and terpenoids. The invention particularly includes a lipophilic phytocannabinoid such as THC or CBD, an excipient fatty acid profile including palmitoleic acid and fatty acids selected from the group consisting of oleic acid and palmitic acid, together with terpenes and terpenoids, and combinations thereof in optimal ratios. Essential oils steam-distilled from plants and other organisms are preferred sources for the terpenes and terpenoids. Macadamia nut oil is a preferred excipient having the desired lipid profile. Macadamia nut oil is a non-toxic plant nut oil, offers oxidation resistance to the fatty acids, cannabinoids and terpenes to improve product shelf life, and has a sufficiently low melting temperature to remain fluid while refrigerated to further enhance product shelf life without changing the product phase from a fluid to a solid.

### FIELD OF THE INVENTION

The present invention relates to the use and formulation of particular lipid and terpene profiles to optimize enteral, oral, transmucosal and transdermal delivery of bioactive compounds. More particularly, the invention relates to the optimization of excipient lipid and terpene profiles including particular fatty acids for delivery of cannabinoids.

### BACKGROUND OF THE INVENTION

Formulations of bioactive compounds have been developed for human and animal consumption and therapeutic use since the beginning of recorded history. Since ancient times flowers and herbs were gathered and consumed therapeutically, often brewed into tea and cooked in food or compressed into a poultice and applied to the skin to treat wounds, pain and other maladies. Animals also self-medicate by eating plants.

Transmucosal and transdermal methodologies often have an advantage over orally deliverable formulations because the human digestive tract can reduce the efficacy and bioavailability of any particular remedy. In particular, first pass metabolism may modify the therapeutic components of orally deliverable bioactive compounds. Additionally stomach acids can inhibit the effectiveness of oral delivery.

Presently, whole-plant botanical mixtures, extracts, decoctions, distillations, essential oils and other various forms have been developed for treating various ailments. Some of these include synthetic and isolated components as well as whole plant extracts. Transdermal patches are commonly used to deliver compounds through the skin.

Oral medications are sometimes specially formulated to absorb directly through the oral mucosa and the oral dermal tissue. Nasal sprays are also formulated to achieve rapid absorption and transmucosal delivery into the bloodstream. Various other delivery modalities have been developed to improve delivery of bioactive compounds.

Transmucosal and transdermal formulations have widely varying pharmacokinetics upon administration depending on the route, manner and system of delivery. Oromucosal and sublingual delivery work well for various bioactive compounds because the digestive tract is bypassed.

With the resurgence of botanical therapeutics in recent years, the vast breadth and depth of therapeutic effects afforded by herbal and traditional systems of medicine and therapies has made the art and science of botanical pharmacokinetics of significant importance.

Human skin includes the epidermis, dermis and subcutis. The epidermis includes the stratum corneum. The stratum corneum is the outer layer of skin that protects the body. It is composed primarily of layers of dead, flattened keratinocytes surrounded by a phospholipid matrix. This can be visualized as brick and mortar wall and functions to provide a barrier to microbial parasites as well as environmental toxins. The stratum corneum also presents a significant barrier to the delivery of transdermal drugs, vitamins, minerals, nutraceuticals and pharmaceuticals. The stratum corneum varies in thickness in various parts of the body. In some regions a mucosal layer covers the skin, such as in the nasal, oral and other bodily cavities. It is commonly intended that bioactive compounds applied to the epidermis of the skin reach the blood vessels of the dermis, entering into general circulation.

There are two major transdermal pathways. One is the intercellular route, which includes the movement of topically applied product around the cells of the stratum corneum via a phospholipid matrix that surrounds the cells. This is a tortuous path, so there are challenges to optimizing the speed of delivery and efficacy of bioactive compounds.

The other pathway is the transcellular pathway, which includes substances directly passing through the cytoplasm of the dead keratinocytes of the stratum corneum, as well as the phospholipids matrix surrounding the cells. This is a more direct pathway yet there are still challenges to optimizing delivery and efficacy of bioactive compounds.

Human mucosa includes outer epithelial layers, superficially keratinized or nonkeratinized, above a basal lamina that separates them from a deeper lamina propria whose papillary layer and capillary plexus contain the blood vessels bioactive compounds are often intended to diffuse into across the superficial mucosa, entering general circulation.

There exists the challenge to penetrate the mucus membranes. Often the mucus membrane is relatively thin, so that the transcellular pathway and the intercellular pathway are more easily navigated when bioactive compounds are delivered transmucosally. This is especially true when an excipient having heightened transmucosal and transdermal diffusion and functionality is formulated with a bioactive compound.

Oleic acid is a common pharmaceutical excipient that is widely used for long-term stabilization, solid formation bulking, and for the therapeutic enhancement of active ingredients including serving as an emulsion agent in topical pharmaceutical formulations and a solubility enhancer for gastrointestinal tract delivery.

What is desired is a formulation including bioactive compounds and a non-toxic excipient carrier that improves rate and efficiency of absorption, bioavailability and efficacy of the bioactive compounds. What is also desired is a formulation having an extended shelf life.

### SUMMARY OF THE INVENTION

The present invention includes a cannabinoid composition including a cannabinoid and an excipient including palmitoleic acid in a therapeutic ratio. The cannabinoid composition includes at least one cannabinoid. In a preferred embodiment, the excipient includes palmitoleic acid, oleic acid and palmitic acid.

The ratio of palmitoleic acid to the at least one cannabinoid is between 1:19 to 1000:1. In another embodiment of the invention, the ratio of palmitic acid to the at least one cannabinoid is between 1:100 to 1000:1. In yet another embodiment of the invention, the ratio of the oleic acid to the at least one cannabinoid is between 1:100 to 1000:1.

In another embodiment of the invention, the ratio of the palmitic acid and oleic acid combined, to the at least one cannabinoid, is between 1:50 to 2000:1. Some research indicates that oleic acid included in the excipient for oral delivery can increase the plasma concentration of bioactive compounds in vivo, as compared to an aqueous excipient. The combination of oleic acid, palmitic acid and palmitoleic acid has also been show to have superior sublingual and oral rates of delivery for cannabinoids.

In an alternate embodiment, the cannabinoid composition is a whole plant extract of Cannabis having at least 60% total cannabinoid content. In another embodiment, the cannabinoid composition is an isolated cannabinoid. In a further embodiment of the invention, the cannabinoid composition is a mixture of isolated cannabinoids having a greater purity than the whole plant extract described in an embodiment of the invention.

In yet another embodiment, the cannabinoid composition includes at least one essential oil selected from the group consisting of Anise, Basil, Bergamot, Cannabis, Cardamom, Chamomile, Cinnamon, Cocoa, Cypress, Eucalyptus, Fennel, Frankincense, Geranium, Ginger, Ginkgo Biloba, Gotu Kola, Grapefruit, Hemp, Jasmine, Lavender, Lavandin, Lemon, Lemon Verbena, Lemongrass, Lime, Marjoram, Neroli, Nutmeg, Orange, Oregano, Peppermint, Pepper, Pine, Ravintsara, Rose, Rosemary, Sage, Spearmint, Tea Tree, Thyme, Turmeric, Vanilla, Ylang Ylang, and any other therapeutic plant essential oils, and combinations thereof, including any of one or more of the monoterpenes, sequiterpenes and/or diterpenes, and/or their alcohols, and/or aldehydes and/or esters normally found in any therapeutic plant essential oil, and combinations thereof.

In one embodiment, the cannabinoid composition includes at least 0.1% by weight of at least one cannabinoid derived from Cannabis sativa L. It can be appreciated that the cannabinoid can be synthetic, derived from yeast, or other plant material.

The cannabinoid composition includes at least 5% by weight of palmitoleic acid. In another embodiment, the invention includes at least one additional fatty acid selected from the group consisting of oleic acid and palmitic acid. In an alternate embodiment the additional fatty acid is any omega-7 fatty acid having synergistic effects for cannabinoid delivery.

In one embodiment of the invention the additional fatty acid is palmitic acid, and the ratio of palmitic acid to the at least one cannabinoid is between 1:100 to 1000:1.

The invention further includes a delivery system. In one embodiment, the delivery system utilizes Transmucosal delivery. Preferably the cannabinoid composition is administered transmucosally in a manner selected from the group consisting of a dropper, spray, swab, lozenge, capsule, gelcap, paste, cream, gel, lotion, ointment, pouch, kitchen utensil, suppository or enema or other manner of transmucosal delivery.

In another embodiment the delivery system includes Oral delivery administered in a manner selected from a dropper, spray, swab, lozenge, capsule, gelcap, emulsion, tincture, kitchen utensil, food, drink or other manner of oral delivery.

In another embodiment the delivery system includes enteral delivery administered in a manner selected from rectal administration of any kind, gastric intubation of any kind, Oral delivery of any kind, or other manner of enteral delivery.

In another embodiment the delivery system includes Transdermal delivery administered in a manner selected from a dropper, spray, swab, patch, paste, cream, gel, lotion or ointment or other manner of transdermal delivery.

The cannabinoid composition consists of any oil or oil blend, whether of a) a single natural origin, b) two or more natural origins, c) one or more fractionated and/or isolated fatty acid(s) and/or glyceride(s), d) one or more synthetic fatty acid(s) and/or glyceride(s), or e) any combination of two or more of (a), (b), (c), or (d); that A) consists of 10% by weight or more of either one, or any mixture of both together, of Macadamia Nut Oil and/or Macadamia-Nut-Oil-simile, and/or B) consists of 10% by weight or more of either one, or any mixture of both together, of Sea Buckthorn Oil and/or Sea-Buckthorn-Oil-simile, and/or C) consists of 5% by weight or more of Palmitoleic Acid.

The cannabinoid composition, in one embodiment, is whole plant Cannabis sativa extract derived from hemp, marijuana, or combinations thereof. Such extracts can be prepared using any of various extraction methods including methods that rely on CO₂, ethanol, hydrocarbon, or combinations thereof. It can be appreciated that other method(s) or solvent(s) can be used for extraction with non-toxic methods preferable.

In the case of the use of an ethanol extraction protocol the concentration of ethanol should be limited to 5000 parts-per-million at the highest, preferably to 50 parts-per-million or less, in the final extract to enable generally safe formulation of the extract with the particular fatty acids expressed herein.

Cannabinoids include one or more of the following: Cannabidiol (CBD), Cannabidiolic Acid (CBDA), Cannabichromene (CBC), Tetrahydrocannabinol (THC), Tetrahydrocannabinolic Acid (THCA), Cannabigerol (CBG), Cannabidivarin (CBDV), Tetrahydrocannabivarin (THCV), or any other compound found exclusively or primarily in the Cannabis sativa L. plant, or any other compound that is a cannabinoid receptor (CB receptor) agonist, co-agonist, partial agonist, inverse agonist or antagonist in the human or animal organism. The sesquiterpene beta-caryophyllene is considered to be a cannabinoid. The cannabinoids can be derived from Cannabis sativa, other plants, manufactured synthetically or derived from animal sources, fungal sources, or combinations thereof.

A cannabinoid is one of a class of diverse chemical compounds that acts on cannabinoid receptors in humans and other animals. The most notable cannabinoid is the phytocannabinoid tetrahydrocannabinol (THC), which is a cannabinoid found in Cannabis sativa. Cannabidiol (CBD) is another major cannabinoid constituent of the plant. There are at least 113 different cannabinoids isolated from Cannabis, each exhibiting varied bioactive effects.

Synthetic cannabinoids encompass a variety of distinct chemical classes: the classical cannabinoids structurally related to Tetrahydrocannabinol (THC), the non-classical cannabinoids (cannabimimetics) including the aminoalkylindoles, 1,5-diarylpyrazoles, quinolines, and arylsulfonamides, as well as eicosanoids related to endocannabinoids.

THC as used herein includes both acid and non-acid forms of this molecule, including isomers thereof. Cannabidiol (CBD) is a major phytocannabinoid found in Cannabis sativa and other plants.

The compositions described herein can be infused with one or more additional bioactive compounds, other than cannabinoids, including botanical essential oil, preparations, and formulations thereof. In another embodiment, the compositions do not include cannabinoids, but instead rely on various non-cannabinoid bioactive compounds, such as essential oils for example.

Essential oils can be derived by steam-distillation, CO2 extraction or any other manner(s) from any one or more plants including, but not limited to, any species or subspecies of any of Anise, Basil, Bergamot, Cannabis, Cardamom, Chamomile, Cinnamon, Cocoa, Cypress, Eucalyptus, Fennel, Frankincense, Geranium, Ginger, Ginkgo Biloba, Gotu Kola, Grapefruit, Hemp, Jasmine, Lavender, Lavandin, Lemon, Lemon Verbena, Lemongrass, Lime, Marjoram, Neroli, Nutmeg, Orange, Oregano, Peppermint, Pepper, Pine, Ravintsara, Rose, Rosemary, Sage, Spearmint, Tea Tree, Thyme, Turmeric, Vanilla or Ylang Ylang.

The Bioactive compound(s) can be one or more of monoterpenes, sequiterpenes and/or diterpenes, and/or their alcohols, and/or aldehydes and/or esters which include, but are not limited to, any of alpha-Bisabolol, alpha-Caryophyllene (also known as Humulene), beta-Caryophyllene, Fenchone, Isobutyl Angelate, Guaiol, Limonene, Linalool, Myrcene, Neral, Nerolidol, Ocimene, alpha-Pinene, beta-Pinene, Terpineol, or Terpinolene, be they a) naturally-present in a botanical extract, or b) an isolate, or c) a synthetic version thereof, or d) a complex or mixture of two or more of any of (a), (b) and/or (c).

In various embodiments, the Bioactive compound includes at least one lipophilic pharmaceutical or nutraceutical compound, other than a cannabinoid. In a variation of the invention various lipophilic nutraceutical compounds including cannabinoids are utilized. In another embodiment, THC, CBD and combinations thereof are utilized as the lipophilic nutraceutical compounds.

In various alternate embodiments, the bioactive compounds include one or more non-lipophilic pharmaceutical or nutraceutical compounds, other than cannabinoids. These non-lipophilic compounds are modified to become soluble in oil by chemical reactions, preferably non-toxic ones, by mixing with at least one lipophilic compound, by emulsification, or by any other process.

It can be appreciated that the bioactive compound can be one or more of a nutritional supplement, vitamin, mineral, and any substance of established or intended macro- or micro-nutritive value to the human or animal organism, and combinations thereof.

In a further embodiment of the invention, the composition described herein includes an aqueous adjuvant such as Aloe Vera, a micro-emulsifier, a nano-emulsifier, ethanol, water, a food, a drink, a substance that has received G.R.A.S. (Generally Recognized As Safe) status by the Food and Drug Administration of the United States of America, a nutrient, a dietary supplement, a nutritional supplement, a botanical extract, a synthetic compound, a natural compound, any combination of any of the previous in any form, or anything else whatsoever.

The combination of excipient fatty acids and components yields a shelf life of up to three years.

### BRIEF DESCRIPTION OF THE DRAWINGS

FIG. 1 is a THC molecule (trans-Δ⁹-tetrahydrocannabinol).

FIG. 2 is a CBD molecule (cannabidiol).

FIG. 3 is an Oleic acid molecule ((9Z)-Octadec-9-enoic acid) having a chemical formula C₁₈H₃₄O₂.

FIG. 4 is a Palmitic acid molecule (Hexadecanoic acid) having a chemical formula C₁₆H₃₂O₂.

FIG. 5 is a Palmitoleic acid molecule ((9Z)-Hexadec-9-enoic acid) having a chemical formula C₁₆H₃₀O₂.

### DETAILED DESCRIPTION

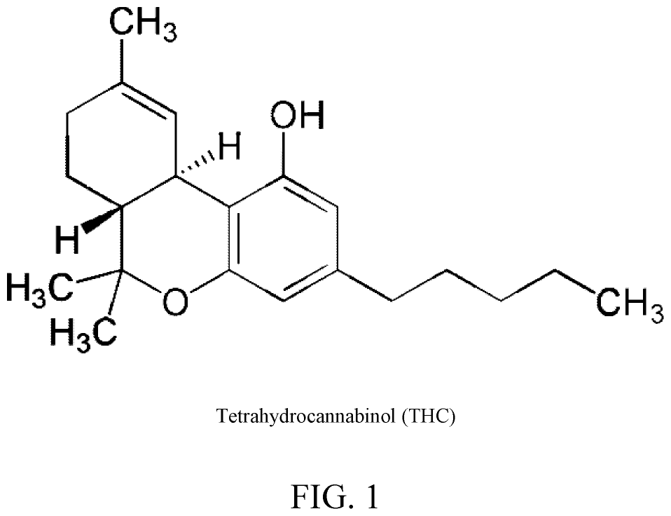

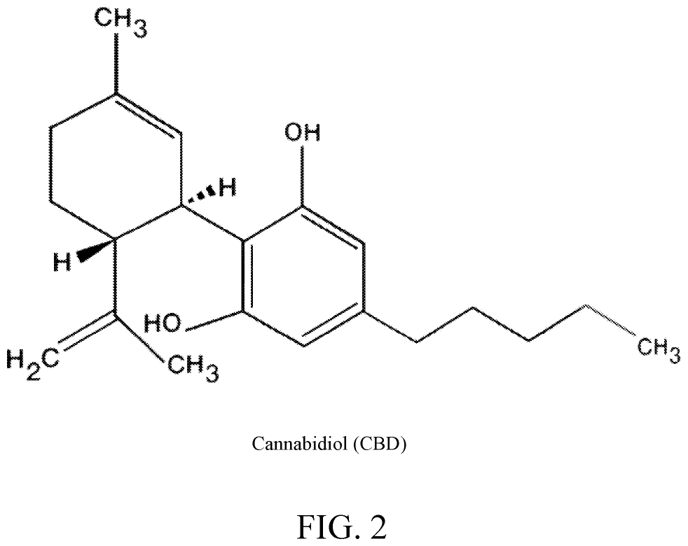

The present invention includes a composition of matter including a cannabinoid and excipient. FIG. 1 and FIG. 2 show THC and CBD molecules, respectively. There are numerous other cannabinoids derived from Cannabis sativa L. that can be substituted in accordance with the present invention.

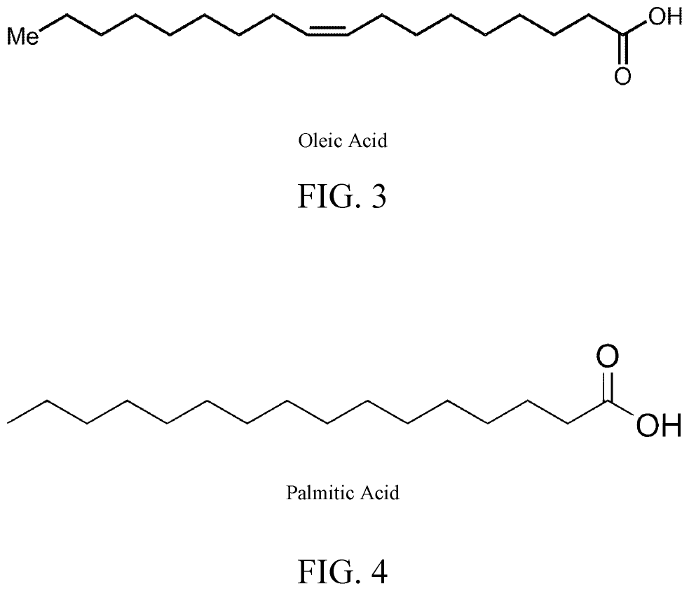

FIG. 3 shows an Oleic acid molecule. FIG. 4 shows a Palmitic acid molecule. Palmitic acid is the most common saturated fatty acid found in animals, plants and microorganisms. Its chemical formula is CH₃(CH₂)₁₄COOH, and its lipid numbers are C16:0. It is a major component of the oil from palm trees (palm oil). It can also be found in various meats, cheeses, butter and dairy products, and used as a food additive. It is understood that some proteins are modified by the addition of a palmitoyl group in a process known as palmitoylation. Palmitoylation is important for membrane localisation of many proteins. It may cooperate with cannabinoids to influence cannabinoid receptor function though the mechanism of action is not fully understood.

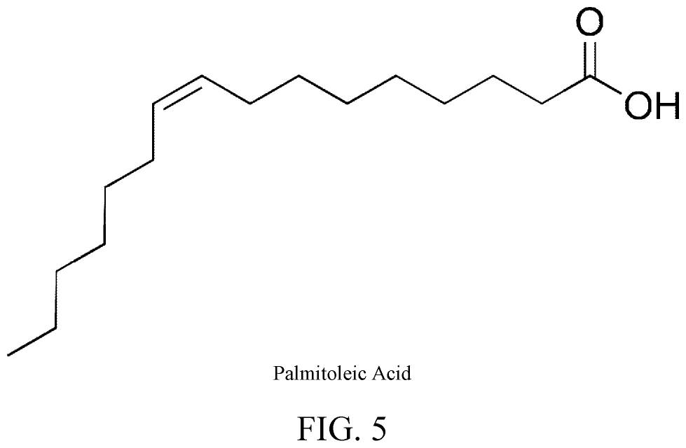

FIG. 5 shows a Palmitoleic acid molecule. Its chemical formula is CH₃(CH₂)₅CH═CH(CH₂)₇COOH. It is present in nearly all human tissues, but is found most concentrated in the liver. In addition to being found in Macadamia oil and sea buckthorn oil, it is also found in the fruit of the durian species Durio graveolens.

In one embodiment of the composition of the present invention, the excipient, or carrier, comprises between 5%-50% palmitoleic acid. In another embodiment, the concentration of palmitoleic acid is greater than 10%. In another embodiment, the palmitoleic acid content of the excipient or carrier, is approximately 15%. The cannabinoid content is preferably at least 1-15% of the composition.

In one embodiment the excipient is Macadamia nut oil, in another embodiment, the excipient is sea buckthorn oil. The excipient further includes oleic acid in a concentration of between 1%-95% and palmitic acid content of 1%-45% by weight. While the use of excipients composed of natural oils is expressed herein, similes and synthetic oils having similar fatty acid profiles can be employed in accordance with the present invention.

In a preferred embodiment, the invention includes a bioactive formulation using the excipient and cannabinoids. The excipient can include a blend of oils, a blend of fractionated oil components, or a blend of natural and synthetic oils including fatty acids. In one embodiment the excipient includes isolated palmitoleic acid and at least one isolated cannabinoid.

In another embodiment the excipient is Macadamia Nut Oil, or a simile thereof.

Table 1 lists some primary fatty acids found in three samples of Macadamia nut oil, M.1, M.2, and M.3. Other studies have revealed that palmitoleic acid concentration can reach to as high as 27% in Macadamia nut oil.

TABLE 1
Composition of tested Fatty Acids in
three samples of Macadamia Nut Oil

| Fatty acid | % (M.1) | % (M.2) | % (M.3) |
|---|---|---|---|
| Myristic | 0.4 | 0.6 | 0.5 |
| Palmitic | 8.4 | 9.5 | 8.8 |
| Palmitoleic | 11.4 | 12.7 | 9.0 |
| Stearic | 5.8 | 3.7 | 7.0 |
| Oleic | 64.7 | 64.8 | 65.1 |
| Linoleic | 2.5 | 1.5 | 1.7 |
| Arachidic | 3.4 | 3.0 | 3.9 |
| Linolenic | 0.2 | 0.2 | 0.1 |
| Behenic | 0.8 | 0.8 | 0.9 |

In another embodiment, the excipient is Sea Buckthorn Oil (Hippophaes rhamnoides). An example of Sea Buckthorn Oil fatty acid composition is shown in Table 2.

TABLE 2
Composition of fatty acids in a sample of sea-buckthorn oil

| | | | | |
|---|---|---|---|---|
| | Content | General | Numerical | Omega |
| Common name | in wt. % | formula | symbol | family |

Saturated fatty acids

| | | | | |
|---|---|---|---|---|
| Palmitic acid | 30-33 | CH₃(CH₂)₁₄COOH | C16:0 | — |
| Stearic acid | <1 | CH₃(CH₂)₁₆COOH | C18:0 | — |

Unsaturated fatty acids

| | | | | |
|---|---|---|---|---|
| Palmitoleic acid | 30-35 | C₁₆H₃₀O₂ | 16:1 | 7 |
| Oleic acid | 14-18 | C₁₈H₃₄O₂ | 18:1 | 9 |
| Linoleic acid | 5-7 | C₁₈H₃₂O₂ | 18:2 | 6 |
| (LA) | | | | |
| α-Linolenic acid | 30 | C₁₈H₃₀O₂ | 18:3 | 3 |
| (ALA) | | | | |
| γ-linolenic acid | 35 | C₁₈H₃₀O₂ | 18:3 | 6 |
| (GLA) | | | | |
| Gondoic acid | 2 | C₂₀H₃₈O₂ | 20:1 | 9 |

There are two traditional ways of testing the lipid and fatty acid content of a sample. Typically gas chromatography (GC) or high-precision liquid chromatography (HPLC) is used. However, these testing methods require a derivatization process to be accurate because of the relatively high boiling points of fatty acids. There are newer derivatization-free gas chromatography (GC) methods for the quantitative analysis of oleic acid and other fatty acids. These newer methods use a nitroterephthalic acid modified polyethylene glycol (PEG) capillary GC column as well as a flame ionization detector (FID). The sample preparation procedure is simpler and does not require derivatization to yield precise results.

Macadamia Nut Oil is a preferred excipient because it is one of the highest in monounsaturated fatty acids of any oil found in nature, which are known to be of high nutritional, preventative and therapeutic value to the healthy function, immunity and repair of the human and animal organisms. Macadamia Nut Oil is also a preferred excipient because it has a high concentration of Oleic Acid. Macadamia Nut Oil is one of few natural oil sources containing over 1% Palmitoleic acid, although sea buckthorn oil can substitute for Macadamia nut oil in accordance with the present invention. Macadamia Nut Oil normally contains over 7% Palmitic Acid. Palmitic acid is considered to be highly polar, which is believed to enhance its capability to effectuate transdermal and transmucosal delivery of bioactive compounds including lipophilic cannabinoids. Macadamia Nut Oil is also a preferred excipient being a natural plant oil known for stability and longevity compared with many other plant oils, particularly oxidation resistance, and normally remains liquid at refrigerator temperatures, allowing it and the compounds it carries to be further preserved in the cold while remaining in a rapidly-administrable liquid state.

Palmitic Acid is the most abundant fatty acid in human tissues overall. Oleic Acid is the most abundant fatty acid in human adipose tissue and the second-most abundant in human tissues overall. Palmitoleic acid is another common constituent of human adipose tissue. Macadamia Nut Oil contains one of the closest natural approximations to the human lipid profile found in the plant kingdom and remarkably yields improved transdermal and transmucosal performance over other plant oils.

Various testing protocols show that Macadamia Nut Oil produces a markedly faster and more intense therapeutic effect upon the subjects than all previously tested carrier oils, particularly when combined with cannabis oil.

Testing and subjective feedback shows that when using the same standardized quantities of the botanical extractions (cannabis oil) and various carrier oils other than Macadamia Nut Oil, bioactivity was detected within 40-120 minutes after Oromucosal administration. Using Macadamia Nut Oil, bioactivity was detected as early as 2 minutes after identical administration protocols were performed. This represents a 20× improvement in the time of first subjectively detected effects with the use of Macadamia nut oil, or an oil having between 5%-27% Palmitoleic Acid, 5%-25% Palmitic Acid, and 50%-70% Oleic Acid as excipient.

Macadamia Nut Oil as an excipient for the delivery of Cannabis oil, plant oils and other essential oils, is a highly-effective, non-toxic, non-invasive, rapid Enteral, Oral, Transmucosal and Transdermal delivery modality for humans. Rapid delivery results when applied sublingually, sublabially or bucally, via the oral mucosal membrane, so the bioactive component begins to be effective within less than 10 minutes, and preferably within two minutes. Subjective data indicates that initial bioactivity resulting from entry of bioactive cannabinoids into the blood stream occurs within 2-10 minutes of administration.

It can be appreciated that the present invention is useful for improved delivery system for human and animal therapeutics alike, with its rapid absorbability and the consequent rapid-acting potential for various bioactive compounds delivered by the invention being of great interest for a wide variety of therapeutic modalities.

Preferably, the present invention includes a bioactive compound such as a whole plant extract of Cannabis sativa in an excipient. The excipient includes a fatty acid selected from the group consisting of oleic acid, palmitic acid, palmitoleic acid, and combinations thereof. Particular ratios of these fatty acids to the bioactive compound are optimal. In one embodiment, the desired ratio of these fatty acids is naturally available through the use of Macadamia Nut Oil as a sole excipient.

Oleic Acid: any isomer of C₁₈H₃₄O₂ (18:1) fatty acid and/or glyceride including, but not limited to, whether naturally-present, fractionated, isolated, or synthesized; or a specific isomer of this fatty acid where specified.

Palmitic Acid: C₁₆H₃₂O₂ (16:0) fatty acid can be derived from any source including naturally occurring, extracted, fractionated, isolated, or synthesized.

Palmitoleic Acid includes any isomer of C₁₆H₃₀O₂ (16:1) and can be derived from a variety of sources and processes, including naturally occurring, extracted fractionated, isolated, or synthesized.

Bioactive compound: a biologically-active compound that exerts a modifying and/or modulating effect upon the human and/or animal organism in any form or modality whatsoever including, but not limited to, the physiological, the psychological, the metabolic, the subjective, the observable, or any other effect whether a Therapeutic Effect or not. This term includes, but is not limited to, botanical extracts, distillations and isolates, nutritional supplements, dietary supplements, and pharmaceutical compounds.

Enteral delivery: relating to entry by any manner into the gastrointestinal tract of the human and/or animal organism(s).

Macadamia Nut Oil: any form of the oil extracted from the seed of any species or subspecies of the Macadamia Tree including, but not limited to, any of Macadamia integrifolia, Macadamia ternifolia and/or Macadamia tetraphylla. This includes the oil in any state or form including, but not limited to, any of natural, cold-pressed, expeller-pressed, virgin, filtered, refined, ethoxylated and/or processed in any form.

Macadamia-Nut-Oil-simile: any oil or oil blend, whether of a) a single natural origin, b) two or more natural origins, c) one or more fractionated and/or isolated fatty acid(s) and/or glyceride(s), d) one or more synthetic fatty acid(s) and/or glyceride(s), or e) any combination of two or more of (a), (b), (c), or (d); that is comprised at least of the fatty acids within the ranges (inclusive of the limits) as follows in Table 3:

TABLE 3
Macadamia-Nut-Oil-simile

| | |
|---|---|
| Oleic Acid (18:1) | 10%-95% |
| Palmitoleic Acid (16:1) | 5%-41% |
| Palmitic Acid (16:0) | 3%-17% |
| Linoleic Acid (18:2) | 0%-16% |
| Stearic Acid (18:0) | 0%-11% |
| Eicosenoic Acid (20:1) | 0%-9% |
| Arachidonic Acid (20:4) | 0%-9% |
| Linolenic Acid (18:3) | 0%-9% |
| Myristic Acid (14:0) | 0%-5% |
| Behenic Acid (22:0) | 0%-5% |
| Lauric Acid (12:0) | 0%-2% |

Oral delivery: relating to entry by the mouth cavity into the gastrointestinal tract of the human and/or animal organism(s).

Oromucosal: relating to the tissues and mucosa that line the oral cavity including, but not limited to, any of the buccal, sublabial, sublingual, pharyngeal and/or laryngeal tissues or cavities of the human and/or animal organism(s).

Rapid Absorption Delivery System (“RAD System”): any oil or oil blend, whether of a) a single natural origin, b) two or more natural origins, c) one or more fractionated and/or isolated fatty acid(s) and/or glyceride(s), d) one or more synthetic fatty acid(s) and/or glyceride(s), or e) any combination of two or more of (a), (b), (c), or (d); that A) consists of 10% by weight or more of either one, or any mixture of both together, of Macadamia Nut Oil and/or Macadamia-Nut-Oil-simile, both as defined in the Definitions, and/or B) consists of 10% by weight or more of either one, or any mixture of both together, of Sea Buckthorn Oil and/or Sea-Buckthorn-Oil-simile, both as defined in the Definitions, and/or C) consists of 5% by weight or more of Palmitoleic Acid; into which is mixed at the least one or more Bioactive compound(s) for administration by the Enteral, Oral, Transmucosal and/or Transdermal route(s) into the human and/or animal organism(s).

Sea Buckthorn Oil: any form of the oil extracted from the pulp of the berry, or from the whole berry, from any species or subspecies of Sea Buckthorn including, but not limited to, any of Hippophae rhamnoides L. subsp. rhamnoides and Hippophae rhamnoides L. subsp. sinensis. This includes any mixture of the oil extracted from the pulp of the berries and the oil extracted from the seed of the berries, or either on its own. This also includes the oil in any state or form including, but not limited to, any of natural, cold-pressed, expeller-pressed, virgin, filtered, refined, ethoxylated and/or processed in any form.

Sea-Buckthorn-Oil-simile: any oil or oil blend, whether of a) a single natural origin, b) two or more natural origins, c) one or more fractionated and/or isolated fatty acid(s) and/or glyceride(s), d) one or more synthetic fatty acid(s) and/or glyceride(s), or e) any combination of two or more of (a), (b), (c), or (d); that is comprised at least of the following fatty acids within the ranges (inclusive of the limits) in Table 4:

TABLE 4
Sea-Buckthorn-Oil-simile

| | |
|---|---|
| Palmitoleic Acid (16:1) | 5%-63% |
| Palmitic Acid (16:0) | 5%-62% |
| Linoleic Acid (18:2) | 0%-40% |
| Oleic Acid (18:1) (cis-9) | 0%-25% |
| Oleic Acid (18:1) (cis-11) | 0%-25% |
| Linolenic Acid (18:3) | 0%-40% |
| Stearic Acid (18:0) | 0%-6% |

Sublabial: relating to the tissues and mucosa behind the lips of the human and/or animal organism(s).

Sublingual: relating to the tissues and mucosa below the tongue of the human and/or animal organism(s).

Therapeutic Effect: having a detectable effect on the physiology or psychology and/or contributing to the sense of well-being of a human or animal. Therapeutic effects can be objective or subjective.

Transdermal: relating to the transport across any part of the skin or dermal membranes of the human and/or animal organism(s).

Transmucosal: relating to the transport across any mucosal membrane of the human and/or animal organism(s) including, but not limited to, any of the buccal, Enteral, Oral, Oromucosal, Sublabial, Sublingual, rectal and/or nasal mucosa.

All ratios expressed herein are on a weight to weight w:w basis unless otherwise stated. Various example formulations are provided as follows:

### Example 1

A tincture including a blend of 10% whole plant cannabis extract and 90% Macadamia nut oil. The whole plant cannabis extract initially has a 60% cannabinoid content. After blending, the tincture has 6% cannabinoid concentration.

### Example 2

A tincture including 18% whole plant cannabis extract and 82% Macadamia nut oil. The cannabis extract has 70% cannabinoid content prior to blending. After blending, the tincture has 12% cannabinoid concentration.

### Example 3

A tincture including blend of 50% hemp flower extract and 50% Macadamia nut oil. The hemp flower extract has a 30% cannabinoid concentration prior to blending. The tincture has a 15% cannabinoid concentration.

### Example 4

A tincture including a blend of pure isolated CBD in a concentration of 15% and 85% Macadamia nut oil.

### Example 5

A tincture including a blend of pure isolated THC in a concentration of 15% and 85% Macadamia nut oil.

### Example 6

A tincture including a blend of pure isolated CBC in a concentration of 10% in 90% Macadamia nut oil.

### Example 7

A tincture including a blend of pure isolated CBN in a concentration of 2% in 98% Macadamia nut oil.

The whole plant Cannabis extract expressed in the examples above is derived from substrate Cannabis plant material, primarily the flowers and leaves. The cannabinoids are directly extracted using conventional extraction methods. While many conventional extraction methods yield between 5-80% cannabinoid content, the present invention encompasses both extracts and concentrates having a cannabinoid content approaching up to 100% cannabinoid content.

Classical cannabinoids expressed herein include all classes derived from cannabigerol-type (CBG) compounds and differ mainly in the way this precursor is cyclized. The classical cannabinoids are derived from their respective 2-carboxylic acids (2-COOH) by decarboxylation (catalyzed by heat, light, or alkaline conditions).

The classical cannabinoids include the following:

THC (Tetrahydrocannabinol)

THCA (Tetrahydrocannabinolic acid)

CBD (Cannabidiol)

CBDA (Cannabidiolic Acid)

CBN (Cannabinol)

CBG (Cannabigerol)

CBC (Cannabichromene)

CBL (Cannabicyclol)

CBV (Cannabivarin)

THCV (Tetrahydrocannabivarin)

CBDV (Cannabidivarin)

CBCV (Cannabichromevarin)

CBGV (Cannabigerovarin)

CBGM (Cannabigerol Monomethyl Ether)

CBE (Cannabielsoin)

CBT (Cannabicitran)

The term “isolate” means a pharmaceutically prepared cannabinoid in a concentration of above 90%, which can then be combined with other cannabinoids in desired ratios. The present invention can utilize desirable isolated cannabinoids to engineer, replace, or supplement, a whole plant cannabinoid extract.

The cannabinoid profile (i.e. ratio of cannabinoids) in the whole plant cannabinoid extract reflects roughly the ratio of cannabinoids in the substrate plant material that is extracted. Whole plant cannabinoid extract does not imply that all parts of the plant including roots and stems are used to create the cannabinoid extract. Preferably, the whole plant cannabinoid extract is manufactured primarily from flowers and leaves of the Cannabis sativa plant.

While the present invention is expressed in conjunction with cannabinoids, the present invention can be utilized to optimize delivery of numerous other bioactive compounds in the manner described, including numerous botanical essential oils.

The excipient can be a broad variety of synthetic and naturally occurring fatty acids, including omega-7 fatty acids. The composition of the present invention is bioactive and can be formulated as a nutraceutical or pharmaceutical composition. Further, “simile” means “analogue” and the terms are interchangeable. Additionally the compositions and formulations of the present invention can be utilized for human consumption as well as administration and consumption by animals including felines and canines.

For added clarity while supplementing the above definitions, with the multiplicity of novel cannabinoid compounds being synthesized for bioactive applications today, the term “classical cannabinoid” as used herein is intended to refer to any compound that can be naturally found within Cannabis sativa L. to be descended from the Cannabigerolic Acid (CBGA) precursor in the plant or from any of its homologs, which includes CBGA, its homologs themselves (e.g., Cannabigerovarinic Acid [CBGVA], Cannnabigerophorolic Acid [CBGPA], Cannabigerobutolic Acid [CBGBA], etc.), and their decarboxylated neutral forms (e.g., Cannabigerol [CBG], Cannabigerovarin [CBGV], Cannabigerophorol [CBGP], Cannabigerobutol [CBGB], etc.), and includes all cannabinoids subsequently biosynthesized or naturally converted within Cannabis sativa L. from any one or more cannabinoids initially derived from CBGA, its homologs, and their decarboxylated neutral forms, whether such cannabinoid gets derived from Cannabis sativa L., another biological source, or is chemically synthesized, and includes any such cannabinoid's isomers, structural analogs, homologs and decarboxylated forms.

Phytoterpenes are a natural class of volatile lipophilic hydrocarbon compounds naturally biosynthesized by plants, as well as other biological sources such as fungi and insects, that can be found concentrated in plant and other organism-derived essential oils. Also known as “isoprenoids”, since they are based upon repeating 5-carbon “isoprene” building blocks of C₅H₈, phytoterpenes fall into four major classes; the monoterpene class having two isoprene units (C₁₀H₁₆), the sesquiterpene class having three isoprene units (C₁₅H₂₄), the diterpene class having four isoprene units (C₂₀H₃₂), and the triterpene class having six isoprene units (C₃₀H₄₈). “Terpenoids” are a modified class of terpenes having additional functional groups, usually oxygen, which include terpene alcohols like the monoterpenoid Linalool and sesquiterpenoid alpha-Bisabolol, terpene ketones like the monoterpenoid Pulegone, terpene aldehydes like the monoterpenoid Citral, and terpene esters like the monoterpenoid Geranyl Acetate. “Phytoterpenes” and “phytoterpenoids” are terpenes and terpenoids, respectively, that are derived from plants.

Phytoterpenes play an important role in plant therapies harnessed for millennia by traditional and classical medicine systems, and are the primary force in aromatherapy. Scientific research reveals phytoterpenes have significant pharmacological activity, being lipophilic, interacting with cell membranes, various ion channels, neurotransmitter receptors and G-protein coupled receptors, such that phytoterpenes have become known as potent therapeutic compounds in their own right. Furthermore, many of these phytoterpenes and the plant essential oils they are found in have been afforded Generally Recognized As Safe (GRAS) status by the United States Food and Drug Administration.

The scientific literature evidence how terpenes can confer significant transdermal penetration enhancement for bioactive compounds across the stratum corneum, due to mechanisms that are the focus of intensive research but have not yet been conclusively established (see Chen et al. 2016). It is proposed that this increased cellular penetration, diffusion and absorption may be mainly due to modifications of the intercellular lipid domain of the stratum corneum by the lipophilic terpenes.

The scientific literature evidence significant absorption enhancements for transdermal bioactive compositions (from 11% increase to over 850% increase in absorption of the target bioactive compound when using terpene concentrations as low as 0.4%-0.5%), when utilizing concentrations of terpenes and ratios of terpenes to the target bioactive compounds well within the ranges covered in the claimed compositions. Taking into account that the stratum corneum provides a significant barrier to bioactive compound permeation, and how its permeation by such compounds is significantly enhanced by the inclusion of terpenes in their bioactive compositions, it becomes clear such significant enhancements might be achieved by terpenes for bioactive compound permeation through less tortuous and more permeable membranes than the stratum corneum, as may be found in the oral mucosa and gastrointestinal mucosa of humans and animals also targeted by this invention.

The scientific literature also evidence that phytoterpenes provide a synergistic enhancing effect on the therapeutic efficacy of phytocannabinoids, which synergy has been included within the famously named “Entourage Effect” that posits a synergistic therapeutic-enhancing effect of combining cannabinoids with non-cannabinoid compounds found in Cannabis. While some recent studies have been published against the “Entourage Effect”, which studies tended to utilize very low doses or a single in vitro model, other recent studies utilizing higher doses (e.g., above 100 mg of terpenes per kg of body weight of animals studied, see LaVigne et al. 2020), indicate not only that terpenes have significant pharmacological activity, but furthermore that they can have agonist activity at the Cannabinoid Receptor Type 1 (CB1), which is also activated by the CB1 partial agonist THC. In addition, human studies analyzing the effects of various phytocannabinoid profiles together with varying phytoterpene profiles found panels of subjects were consistently experiencing specific types of subjective effects in relation to specific phytoterpene profiles (see Lewis et al. 2018).

Modern essential oil safety research has determined that a safe upper limit for oral ingestion of essential oils derived from safe, nontoxic edible plants is normally 2% on a weight to weight basis (see Tisserand & Young 2016). It is therefore suggested that a 2% w:w upper safety limit for the total combined terpene and terpenoid concentration in an oral or oromucosal composition not be significantly exceeded. Depending on the terpenes used, a 10% maximum concentration for safety of dermal terpene application is often seen, and the use of lower concentrations is advised.

Taking into consideration further safety concerns, it is advised to only utilize terpenes or essential oils that have been extracted using nontoxic methods, with steam distillation being ideal, while subcritical or supercritical carbon dioxide extraction is also feasible, though less effective than steam distillation for selectively isolating just the terpenes and terpenoids from the source biomass. Steam distillation is preferred for its ability to produce a very high-purity essential oil nearing 100% concentration of plant volatiles, being primarily comprised of terpenes and terpenoids, without any residual solvents or non-volatile constituents. Steam distillation of plant essential oils is an artform dating back several centuries, which has also now been refined so that varying spectra of a plant's essential oil constituents can be targeted in the final essential oil extracted by varying the temperature and intensity of the steam utilized, as well as by extracting essential oil distilled during specific windows of time during the steam distillation process. The purity of the source plant biomass used for the extraction of terpenes and terpenoids is also important, ideally farmed using organic methods and tested before extraction for pesticides, toxic metals, microbiology including bacteria, yeast and mold, and mycotoxins, to eliminate toxic pesticide residues, toxic metals, microbes, mold spores, or mycotoxins from entering and adversely affecting the target organism's health via a tainted extract.

It can therefore be appreciated that a lipophilic bioactive composition, whether including cannabinoids or not, can have its cellular permeation, pharmacokinetic profile, and bioactive pharmacodynamic profile enhanced by including high-purity terpenes in the composition at effective concentrations and ratios to the target bioactive compounds.

Accordingly, various subjective testing protocols using the claimed compositions in oromucosal and oral ingestion trials showed that the inclusion of various terpene profiles within cannabinoid compositions reduced the time to onset of subjective therapeutic effect, and increased the intensity of subjective therapeutic effect, according with the reports in the scientific literature. Particularly for the reduction of pain, reduction of anxiety, and reduction of insomnia, as the terpene-to-cannabinoid ratio of the formulation was increased, so did the antinociceptive, anxiolytic, and somnolent effects of the formulation increase. Such formulations were created by taking the cannabinoid compositions and adding either single terpene isolates such as beta-Myrcene, Linalool, alpha-Pinene, Menthol, or beta-Caryophyllene, for example, or adding multiple terpene isolates combined to partially reconstruct naturally-occurring plant terpene profiles as determined from gas chromatography testing of Cannabis and other plant essential oils, or adding natural essential oils derived from various Cannabis varieties, including hemp varieties, and other plants such as lavender, frankincense, chamomile and rose, for example. All the while, for oromucosal safety a maximum 2.5% w/w of total terpene content in the final trial oral formulations was not breached, comprised of the added terpenes together with the terpenes naturally found in the cannabinoid extract used. Notably, improved time-to-onset and intensity of therapeutic effects were subjectively reported when adding terpenes with formulation ratios of cannabinoids to terpenes approaching 1000:1 and lower.

It can therefore be appreciated that the combination of the excipient fatty acids in the claimed compositions of this invention with the terpenes and terpenoids in the claimed compositions of this invention produces a significant synergistic increase in the absorption and efficacy of the bioactive compounds in these claimed compositions, in a novel and inventive manner not seen when either part or parts are utilized alone.

All ratios expressed herein are on a weight to weight w:w basis unless otherwise stated. Adding to Examples 1-7 above, various example formulations including cannabinoids, terpenes and Macadamia nut oil for maximal bioavailability and bioactivity are provided as follows:

### Example 8

A tincture including 18% cannabis flower extract, 81.9% Macadamia nut oil and 0.15% cannabis essential oil. The cannabis flower extract has 70% cannabinoid content and 2% combined terpene and terpenoid content prior to blending. The cannabis essential oil has 98% terpene and terpenoid content prior to blending. After blending, the tincture has 12.6% cannabinoid concentration and 0.5% concentration of terpenes and terpenoids combined, resulting in a cannabinoid-to-terpene ratio of 25:1.

### Example 9

A tincture administered via dropper including 10% hemp plant extract, 89.7% Macadamia nut oil and 0.3% hemp essential oil. The hemp plant extract has 60% cannabinoid content and 1% combined terpene and terpenoid content prior to blending. The hemp essential oil has 98% terpene and terpenoid content prior to blending. After blending, the tincture has 6% cannabinoid concentration and 0.4% concentration of terpenes and terpenoids combined, resulting in a cannabinoid-to-terpene ratio of 15:1.

### Example 10

A topical oil including 16.7% hemp plant extract, 80.9% Macadamia nut oil and 2.4% of monoterpenoid Menthol isolate. The hemp plant extract has 60% cannabinoid content and 1% combined terpene and terpenoid content prior to blending. The Menthol isolate is 98% in purity prior to blending. After blending, the topical oil has 10% cannabinoid concentration and 2.5% concentration of terpenes and terpenoids combined, which terpene and terpenoid fraction is 94% comprised of Menthol, resulting in a cannabinoid-to-terpene ratio of 4:1.

### Example 11

An oral oil administered using a needle-less syringe including 30% hemp plant extract, 69.9% Macadamia nut oil and 0.1% of a terpene isolates blend. The hemp plant extract has 75% cannabinoid content and 1% combined terpene and terpenoid content prior to blending. The terpene isolates blend has 95% terpene and terpenoid content prior to blending. After blending, the oral oil has 22.5% cannabinoid concentration and 0.4% concentration of terpenes and terpenoids combined, resulting in a cannabinoid-to-terpene ratio of 57:1.

### Example 12

An oil capsule including 17% hemp plant distillate, 80.4% Macadamia nut oil and 2.6% of monoterpenoid Linalool isolate. The hemp plant distillate has 90% cannabinoid content and 0.1% combined terpene and terpenoid content prior to blending. The Linalool isolate is 95% in purity prior to blending. After blending, the oil capsule has 15% cannabinoid concentration and 2.5% concentration of terpenes and terpenoids combined, which terpene and terpenoid fraction is 99% comprised of Linalool, resulting in a cannabinoid-to-terpene ratio of 6:1.

### Example 13

An oromucosal oil spray including 7.5% CBD isolate, 7.5% CBG isolate, 84.3% Macadamia nut oil and 0.75% of mandarin essential oil. The CBD and CBG isolates have 99% purity and no detected terpene and terpenoid content prior to blending. The mandarin essential oil has 98% combined terpene and terpenoid content prior to blending. After blending, the oromucosal oil spray has 15% cannabinoid concentration and 0.74% concentration of terpenes and terpenoids combined, resulting in a cannabinoid-to-terpene ratio of 20:1.

Since the term “terpenoid” is normally used to refer to a class of modified terpenes, it is herein usually used separately to the term “terpene”, even though the two terms “terpene” and “terpenoid” may mutually be used interchangeably to refer to one another.

1-16. (canceled)

17. A bioactive composition comprising:
at least one nutraceutical compound;
palmitoleic acid, wherein the ratio of palmitoleic acid to the at least one nutraceutical compound in the bioactive composition is between 1:19 to 1000:1 on a weight to weight basis;
palmitic acid, wherein the ratio of palmitic acid to the at least one nutraceutical compound in the bioactive composition is between 1:100 to 1000:1 on a weight to weight basis;
oleic acid, wherein the ratio of the oleic acid to the at least one nutraceutical compound in the bioactive composition is between 1:100 to 1000:1 on a weight to weight basis; and
at least one terpene or terpenoid in a concentration of at most 10% of the bioactive composition on a weight to weight basis, wherein the ratio of the at least one terpene or terpenoid to the at least one nutraceutical compound is at least 1:1000 on a weight to weight basis.

18-19. (canceled)

20. A bioactive composition comprising:
at least one pharmaceutical compound;
palmitoleic acid, wherein the ratio of palmitoleic acid to the at least one pharmaceutical compound in the bioactive composition is between 1:19 to 1000:1 on a weight to weight basis;
palmitic acid, wherein the ratio of palmitic acid to the at least one pharmaceutical compound in the bioactive composition is between 1:100 to 1000:1 on a weight to weight basis;
oleic acid, wherein the ratio of the oleic acid to the at least one pharmaceutical compound in the bioactive composition is between 1:100 to 1000:1 on a weight to weight basis; and
at least one terpene or terpenoid in a concentration of at most 10% of the bioactive composition on a weight to weight basis, wherein the ratio of the at least one terpene or terpenoid to the at least one pharmaceutical compound is at least 1:1000 on a weight to weight basis.
